(19) United States
(12) Patent Application Publication
(10) Pub. No.: US 2020/0078425 A1
(43) Pub. Date: Mar. 12, 2020

(54) Methods and Compositions for Treating Obesity, Inflammation, or Metabolic Disorders with Bacteriophages

(71) Applicant: Brigham Young University, Provo, UT (US)

(72) Inventors: Laura Clarke Bridgewater, Provo, UT (US); Gongze Zhao, Highland, UT (US)

(73) Assignee: Brigham Young University, Provo, UT (US)

(21) Appl. No.: 16/493,975

(22) PCT Filed: Mar. 14, 2018
(86) PCT No.: PCT/US2018/022419

§ 371 Date: Sep. 13, 2019

Related U.S. Application Data
(60) Provisional application No. 62/471,248, filed on Mar. 14, 2017.

(51) Int. Cl. A61K 35/76; A61P 3/04; A61P 29/00

(52) U.S. Cl. CPC A61K 35/76; C12N 2795/00032; A61P 29/00; A61P 3/04

(57) ABSTRACT

The present disclosure as disclosed in various embodiments relates to methods and compositions of treating obesity, inflammation, or obesity-associated metabolic disorders with bacteriophages and processes for preparing the compositions.

### CROSS-REFERENCE TO RELATED APPLICATIONS

This application claims the benefit of U.S. Provisional Application No. 62/471,248 filed Mar. 14, 2017, the disclosure of which is incorporated in its entirety by reference herein.

### TECHNICAL FIELD

The disclosure generally relates to methods and compositions of treating obesity, inflammation, or obesity-associated metabolic disorders with bacteriophages and processes for preparing the compositions.

### BACKGROUND

Obesity has become an epidemic worldwide. Overweight and obesity rates in the U.S. have increased dramatically over the past few decades, culminating in 37% of Americans (i.e. 120 million people) qualifying as obese². In the United States the prevalence of obesity was 35% among men and 40% among women in 2013 to 2014². Six percent of American adults are morbidly obese, which is a 400% increase since 1986^{9,10}. Among children and adolescents, the prevalence of obesity was 17% in 2010⁷. Obesity is often the major contributor to many other chronic illnesses such as diabetes, heart attacks, strokes, and certain cancers³. Obesity contributes to heart disease, fatty liver, osteoarthritis, depression, and certain cancers, which result in diminished quality of life and skyrocketing healthcare costs^{4,5}.

### SUMMARY

The present disclosure as disclosed in various embodiments relates to methods and compositions of treating obesity, inflammation, or obesity-associated metabolic disorders with bacteriophages and processes for preparing the compositions.

In various embodiments are disclosed compositions for altering an intestinal microbiome of a subject to treat obesity, inflammation, or obesity-associated metabolic disorders including an amount of a bacteriophage strain effective for reducing a concentration of a pathogen in an intestinal microbiome of a subject and a pharmaceutically acceptable excipient, wherein the concentration of the pathogen induces obesity, inflammation, or an obesity-associated metabolic disorder and the bacteriophage strain has an infectivity specific to a species of the pathogen. In various embodiments are disclosed processes of preparing compositions of various embodiments including: characterizing the bacteriophage strain by exposing the pathogen to the bacteriophage strain and detecting infection or lysis of the pathogen; and combining the bacteriophage strain with the pharmaceutically acceptable excipient when infection or lysis of the pathogen is detected.

In various embodiments are disclosed methods for altering an intestinal microbiome of a subject to treat obesity, inflammation, or obesity-associated metabolic disorders, the method including: administering an amount of a bacteriophage strain to a subject with an intestinal microbiome having a concentration of a pathogen inducing obesity, inflammation, or an obesity-associated metabolic disorder, wherein the amount of a bacteriophage strain reduces the concentration of the pathogen and the bacteriophage strain has an infectivity specific to a species of the pathogen.

In various embodiments are disclosed processes for preparing a composition for altering an intestinal microbiome of a subject to treat obesity, inflammation, or obesity-associated metabolic disorders, the process including: identifying a pathogen, where a concentration of the pathogen in the intestinal microbiome of the subject induces obesity, inflammation, or an obesity-associated metabolic disorder; characterizing a bacteriophage strain by exposing the pathogen to the bacteriophage strain and detecting infection or lysis of the pathogen; and preparing a composition for altering the intestinal microbiome of a subject to treat obesity, inflammation, or an obesity-associated metabolic disorder by combining the bacteriophage strain with a pharmaceutically acceptable excipient when infection or lysis of the pathogen is detected.

### BRIEF DESCRIPTION OF THE DRAWINGS

For a further understanding of the nature, objects, and advantages of the present disclosure, reference should be had to the following detailed description, read in conjunction with the following drawings, wherein:

FIGS. 1A, 1B, 1C, 1D, 1E, 1F, 1G, 1H, and 1I are graphical representations showing lytic activity of different bacteriophage strains of various embodiments on Enterobacter cloacae strain B29.

FIG. 2 is a graphical representation showing arrested weight gain in mice with intestinal microbiomes colonized with Enterobacter cloacae strain B29 treated with bacteriophages as compared to control mice with intestinal microbiomes colonized with Enterobacter cloacae strain B29.

FIG. 3 is a graphical representation showing a study designed to measure the efficacy of phage treatment when administered before exposure to Enterobacter cloacae strain B29, after exposure to Enterobacter cloacae strain B29 but before obesity develops, and after obesity develops due to colonization of the gut microbiota by Enterobacter cloacae strain B29.

### DETAILED DESCRIPTION

As required, detailed embodiments of the present disclosure are disclosed herein; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “plurality” and “at least two” are used interchangeably.

The term “pathogen” is understood to mean any microorganism, such as bacteria, fungae, or protists, capable of becoming a part of a microbiome of a subject and capable of inducing obesity, inflammation, and/or obesity-associated metabolic disorders when part of the microbiome.

The term “microbiome” refers to the totality of microbes (bacteria, fungae, protists), their genetic elements (genomes) in a defined environment. The microbiome may be a gut microbiome (i.e. intestinal microbiome).

The terms “bacteriophage” and “phage” are used interchangeably and can include naturally-occurring and recombinant bacteriophages, unless otherwise indicated. Alternatively, the term “phage” can refer to viruses capable of infecting fungae or protists. A “naturally-occurring” bacteriophage is a phage isolated from a natural or human-made environment that has not been modified by genetic engineering. A “recombinant bacteriophage” is a phage that comprises a genome that has been genetically modified by insertion of a heterologous nucleic acid sequence into the genome or removal of a nucleic acid sequence from the genome. In some embodiments, the genome of a naturally-occurring phage is modified by recombinant DNA technology to introduce a heterologous nucleic acid sequence into the genome at a defined site. In other embodiments, the genome of a naturally-occurring phage is modified by recombinant DNA technology to remove nucleic acid sequences that, for example, encode toxins.

The term “strain(s)” refers to either microorganism(s) or bacteriophage(s), has its conventional meaning as used in the art, that is, generally, a low taxonomic rank indicating a genetic variant or subtype of a microorganism (within a defined species) or of bacteriophage (within a defined species). Most likely, but not necessarily, bacteriophage of a different strain will have varying specificity with regard to the infection of different strains of bacteria.

The terms “polynucleotide”, “nucleotide”, or “nucleotide sequence” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components.

The terms “peptide” or “protein” as used herein refers to any peptide, oligopeptide, polypeptide, gene product, expression product, or protein. A peptide is comprised of consecutive amino acids. The terms “peptide” or “protein” encompass naturally occurring or synthetic molecules.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “treating” refers to decreasing in one or more symptoms characteristic of a disease or disorder; a decrease in the rate of progression of the disease or disorder; recovery from the disease or disorder, cure from the disease or disorder, maintenance of remission and prophylaxis such as prevention of relapse.

The term “subject(s)” refers to subjects of any mammalian subject(s) of any mammalian species such as, but not limited to, humans, dogs, cats, horses, rodents, any domesticated animal, or any wild animal.

The term “‘#’ pathogen”, where “#” includes, for example, first, second, third, or fourth, can be understood to mean “‘#’ or more different pathogens”. For example, fourth pathogen can be understood to mean four or more different pathogens.

The present disclosure as disclosed in various embodiments relates to methods and compositions of treating obesity, inflammation, or obesity-associated metabolic disorders with bacteriophages and processes for preparing the compositions. Bacteriophages are intracellular parasites that multiply inside bacteria by making use of some or all of the host biosynthetic machinery. Bacteriophages generally contain nucleic acid and protein, and may be covered by a lipid membrane. A description of bacteriophages can be found in U.S. Pat. No. 9,617,522, the disclosure of which is incorporated in its entirety by reference herein.

Recent research has revealed that dysbiosis (imbalance) of the gut microbiota fosters obesity^{11,12}, and causal bacterial strains are being identified. They appear to promote obesity by increasing low-level inflammation throughout the body, which impairs insulin responsiveness^{13,14,15}. Some may also increase the harvest and storage of energy from food. The gram-negative opportunistic pathogen Enterobacter cloacae strain B29, isolated from the gut of an obese human, was the first human gut bacteria shown to cause obesity when transplanted into germ-free mice¹. Particularly, research using Koch's postulates showed that Enterobacter cloacae strain B29 causes obesity and chronic inflammation in its host. Research has also shown that the concentration of Clostridium ramosum in the gut microbiota of obese humans^{16,17} and women with type 2 diabetes¹⁸ are elevated. Clostridium ramosum, when transplanted into germ-free mice, also induced obesity¹⁹. Further research related to two more strains of obesogenic human gut bacteria is currently being undertaken. Research has also shown rapid weight regain after diet-induced weight loss, due to persistence of these obese-type gut microbiota²⁰. This research²⁰ highlights that the composition of the gut microbiota is resistant to change. Dieting can lead to temporary weight loss without producing structural changes in the gut microbiota. Probiotic foods and pills are also unlikely to produce lasting changes, because bacterial species that are already stably established in the gut ecosystem have a strong advantage over newly introduced species.

To address these problems, the present disclosure as described in varying embodiments targets and eliminates harmful obesity-promoting (obesogenic) gut microbes. By eliminating these gut microbes, ecological space is created for beneficial bacteria to thrive. This improves the balance of the gut microbiota. Along these lines, the present disclosure as described in varying embodiments could, for example, be marketed as a nutritional supplement.

The compositions and methods of various embodiments include bacteriophages. Bacteriophages (phages) are viruses that infect bacteria by binding at specific and unique binding sites on the cell surface. This ability of phage to kill bacteria specifically while not infecting human cells or killing non-target bacteria makes them a potential alternative for eliminating pathogenic bacteria such as Enterobacter cloacae B29 or Clostridium ramosum from the gut to create more ecological space so beneficial bacteria can flourish. Unlike broad-spectrum antibiotics, each phage only kills specific bacteria, making it possible to eradicate only the pathogenic bacteria (like Enterobacter cloacae B29 or Clostridium ramosum) in the gut while leaving the beneficial bacteria (probiotics) to flourish, thus treating obesity, inflammation, and/or obesity associated metabolic disorders.

In various embodiments are disclosed compositions for altering an intestinal microbiome of a subject to treat obesity, inflammation, or obesity-associated metabolic disorders including an amount of a bacteriophage strain effective for reducing a concentration of a pathogen in an intestinal microbiome of a subject and a pharmaceutically acceptable excipient, wherein the concentration of the pathogen induces obesity, inflammation, or an obesity-associated metabolic disorder and the bacteriophage strain has an infectivity specific to a species of the pathogen. The amount of bacteriophage strain of various embodiments is effective for reducing a concentration of endotoxins produced by the pathogen in the intestinal microbiome of the subject.

In various embodiments, the pathogen is capable of inducing obesity, inflammation, or an obesity-associated metabolic disorder. Examples of pathogens of various embodiments include pathogens belonging to a genus Enterobacter such as Enterobacter cloacae strain B29 or pathogens belonging to a genus Clostridium including pathogens such as Clostridium ramosum. The pathogen of various embodiments may produce endotoxins such as lipopolysaccharides that induces obesity, inflammation, or an obesity-associated metabolic disorder, or the pathogen may increase the harvest or storage of energy from food.

Examples of bacteriophages of various embodiments include Myoviridae (T4-like virus; P1-like viruses; P2-like viruses; Mu-like viruses; SPO1-like viruses; phiH-like viruses); Siphoviridae (λ-like viruses, γ-like viruses, T1-like viruses; T5-like viruses; c2-like viruses; L5-like viruses; .psi.M1-like viruses; phiC31-like viruses; N15-like viruses); Podoviridae (T7-like virus; phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus), Cystoviridae (Cystovirus), Ampullaviridae, Bicaudaviridae, Clavaviridae, Globuloviridae, and Guttavirus. In various embodiments, the bacteriophage strain is unable to infect other microorganisms or cells other than the pathogen or other pathogens from the species of the pathogen.

In various embodiments, the bacteriophage reduces the concentration of the pathogen by infecting and lysing the pathogen. The infecting of various embodiments is accomplished through infecting a binding site or surface receptor unique to the pathogen or the species of the pathogen.

The bacteriophage strain of various embodiments is a lytic phage to the pathogen that when infected does not develop resistances to the bacteriophage strain for a time or more than the time. In various embodiments, the time includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, or indefinitely. In various embodiments, the time is selected between any two times from above.

The bacteriophage strain of various embodiments does not have genes encoding bacterial toxins and/or virulence factors including, for example, cholerae toxin, botulinum toxin, and diphtheria toxin. The toxins or virulence factors includes toxins or virulence factors associated with, for example, diphtheria, cholera, dysentery, botulism, food poisoning, scalded skin syndrome, necrotizing pneumonia or scarlet fever. The bacteriophage strain of various embodiments is a mutant or a recombinant bacteriophage strain of an isolated bacteriophage strain capable of infecting and lysing the pathogen. The mutant of various embodiments is prepared, for example, by exposing an isolated bacteriophage strain to a mutagenic agent such as a chemical mutagen or electromagnetic radiation. Recombinant bacteriophage strains of various embodiments is prepared by inserting a polynucleotide into the genome of the isolated bacteriophage strain, where the polynucleotide encodes for an antibacterial protein or therapeutic protein. Examples of antibacterial and therapeutic proteins are disclosed in PCT Application Publication No. WO 2018/174810 and WO 2018/030323, the disclosures of which are incorporated in its entirety by reference herein. In another example, the bacteriophage strain of various embodiments has a genome that is not more than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an isolated bacteriophage strain such as Myoviridae PG7. The percent similarity of the genome of the bacteriophage of various embodiments to the isolated bacteriophage strain is selected between any two percentages from above.

The bacteriophage strain of various embodiments is stable for incorporation to various food and pharmaceutical compositions and are efficacious to reach and infect microorganisms such as Enterobacter cloacae colonized in the gut of a subject such as, for example, humans, canines, felines, etc. In various embodiments, the amount of the bacteriophage strain includes, includes more than, or includes less than 10², 10³, 10⁴, 10⁵, 10⁶, 10⁷, 10⁸, 10⁹, 10¹⁰, 10¹¹, 10¹², 1013, 10¹⁴, 10¹⁵, 10¹⁶, 10¹⁷, 10¹⁸, 10¹⁹, and 10²⁰ plaque forming units per gram or milliliter of the various compositions. In various embodiments, the plaque forming units per gram or milliliter is selected between any two points from above.

In various embodiments, the bacteriophage strain is prepared in any manner to be incorporated as a component of a pharmaceutical, food stuff, feed additive, or liquid additive and can be in various forms such as, for example, a liquid state or a dried state including as a powder. In other examples, the bacteriophage strain of various embodiments is dried by air drying method, natural drying method, a spray drying method, a freeze-drying method, or the like. The preparation of the bacteriophage strain can also serve to enhance the properties of the composition including stability. The term “foodstuff” is understood to be any substance or product which in the processed, partially processed, or unprocessed state are intended to be, or reasonably expected to be, ingested by humans. “Foodstuff” can also include drinks, chewing gum, and any substance—including water-intentionally added to the foodstuff during its manufacture, preparation or treatment. The term “feed” is understood to cover all forms of animal food. Foodstuffs can also be used as feeds. The term “pharmaceutical” is understood to cover substances or substance compositions which are intended as agents having properties for curing or for preventing human or animal diseases or which can be used in or on the human or animal body or administered to a human or animal in order to restore, correct or influence either human or animal physiological functions by a pharmacological, immunological or metabolic action, or to produce a medical diagnosis. Pharmaceuticals can be used for non-therapeutic, in particular cosmetic, purposes.

In various embodiments are disclosed pharmaceutical compositions including a compound of any embodiments wherein the pharmaceutical composition is a gel capsule, tablet, pill, lozenge, capsule, microcapsule, liquid, or syrup.

In various embodiments are disclosed orally consumable products including a composition of any embodiment, wherein an orally consumable product is a semi-solid food, solid food, a semi-solid or solid spoonable food, confectionary, drink, or dairy product. The dairy product of various embodiments is ice cream, milk, milk powder, yogurt, kefir, or quark.

In various embodiments are disclosed processes of preparing compositions of various embodiments including: characterizing the bacteriophage strain by exposing the pathogen to the bacteriophage strain and detecting infection or lysis of the pathogen; and combining the bacteriophage strain with the pharmaceutically acceptable excipient when infection or lysis of the pathogen is detected. In various embodiments, the characterizing includes exposing a second pathogen from the species of the pathogen to the bacteriophage strain and detecting infection or lysis of the second pathogen and the combining includes combining the bacteriophage strain with the pharmaceutically acceptable excipient when infection or lysis of the second pathogen by the bacteriophage strain is detected. In other embodiments, the characterizing includes exposing a second pathogen of a species different from the species of the pathogen with the bacteriophage strain and detecting infection or lysis of the second pathogen and the combining includes combining the bacteriophage strain with the pharmaceutically acceptable excipient when infection or lysis of the second pathogen by the bacteriophage strain is not detected. For example, the bacteriophage strain is characterized using a pathogen and a beneficial microorganism. Further, the second pathogen is understood to be at least two different pathogens.

The compositions of various embodiments when administered is used to reduce concentrations of different and multiple pathogens strains, species of pathogens, and genus of pathogens. The compositions of various embodiments include multiple bacteriophage strains for a pathogen or species of a pathogen or multiple different pathogens.

In various embodiments, the composition includes at least two different bacteriophage strains, where each bacteriophage strain has an infectivity specific to the species of the pathogen. In various embodiments, the number of different bacteriophage strains include 2, 3, 4, 5, 6, 7, 8, 9, 10, 11, 12, 13, 14, 15, 16, 17, 18, 19, and 20 different bacteriophage strains. In various embodiments, the number of different bacteriophage strains is selected between any two points from above. For the at least two different bacteriophage strains of various embodiments, each bacteriophage strain has a genome with less than 100% nucleotide sequence identity to genome(s) of the other bacteriophage strain(s) or 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% nucleotide sequence identity to genome(s) of the other bacteriophage strain(s). In various embodiments, nucleotide sequence identity is a range between any two percentages listed above.

In various embodiments are disclosed processes of preparing compositions with at least two different bacteriophage strains including: characterizing bacteriophage strains by separately exposing the pathogen to each bacteriophage strain and detecting infection or lysis of the pathogen; selecting the at least two different bacteriophage strains from the bacteriophage strains that infect or lyse the pathogen; and combining the at least two different bacteriophage strains with the pharmaceutically acceptable excipient.

In various embodiments, the composition includes an amount of a second bacteriophage strain effective for reducing a concentration of a second pathogen of a species different from the species of the pathogen and in the intestinal microbiome of the subject, wherein the second pathogen induces obesity, inflammation, or an obesity-associated metabolic disorder and the second bacteriophage strain has an infectivity specific to a species of the second pathogen. An amount of a second bacteriophage strain is also understood to mean compositions of various embodiments having amounts of multiple bacteriophage strains with each of the multiple bacteriophage strains having infectivity specific to different pathogens and species of the different pathogens. For example, the compositions have amounts of or more than 1, 2, 3, 4, 5, 6, 7, or 8 different bacteriophage strains, where each bacteriophage strain has an infectivity specific to different pathogens and species of the different pathogens. In various embodiments, the number of bacteriophage strain specific for a different pathogen is a range between any two numbers listed above. Also, each bacteriophage strain specific for a different pathogen of various embodiments includes at least two or 2, 3, 4, 5, 6, 7, 8, 9, 10, 11, 12, 13, 14, 15, 16, 17, 18, 19, or 20 different bacteriophage strains. In various embodiments, the number of different bacteriophage strains specific to a pathogen is a range between any two numbers listed above. In various embodiments, each bacteriophage strain has a genome with less than 100% nucleotide sequence identity to genome(s) of the other bacteriophage strain(s) or 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% nucleotide sequence identity to genome(s) of the other bacteriophage strain(s). In various embodiments, nucleotide sequence identity is a range between any two percentages listed above.

The composition of various embodiments is a cocktail(s) of bacteriophage strains specific for species of a first pathogen, second pathogen, third pathogen, and a fourth pathogen, where each pathogen is different from the others. For example, the first pathogen could be from the species Enterobacter cloacae and the second pathogen could be from the species Clostridium ramosum. It is understood that the fourth pathogen is understood to mean four or more different pathogens. Also for each pathogen, the number of different bacteriophage strains includes 1, 2, 3, 4, 5, 6, 7, 8, 9, 10, 11, 12, 13, 14, 15, 16, 17, 18, 19, or 20 different bacteriophage strains. In one example, the composition includes: a cocktail with 1 to 20 different bacteriophage strains having an infectivity specific for a species of a first pathogen; a cocktail with 1 to 20 different bacteriophage strains having an infectivity specific for a species of a second pathogen; a cocktail with 1 to 20 different bacteriophage strains having an infectivity specific for a species of a third pathogen; a cocktail with 1 to 20 different bacteriophage strains having an infectivity specific for a species of a fourth pathogen; and, optionally, additional cocktail(s) with 1 to 20 different bacteriophage strains having an infectivity or infectivities specific for species of additional pathogen(s).

The following as disclosed in various embodiments highlight compositions with different bacteriophage strains.

In various embodiments, the composition includes an amount of a second bacteriophage strain effective for reducing a concentration of a second pathogen of a species different from the species of the pathogen and in the intestinal microbiome of the subject, wherein the second pathogen induces obesity, inflammation, or an obesity-associated metabolic disorder and the second bacteriophage strain has an infectivity specific to a species of the second pathogen. In various embodiments are disclosed processes of preparing compositions of various embodiments including: characterizing the bacteriophage strain by exposing the pathogen to the bacteriophage strain and detecting infection or lysis of the pathogen; characterizing the second bacteriophage strain by exposing the second pathogen to the second bacteriophage strain and detecting infection or lysis of the second pathogen; and combining the bacteriophage strain and the second bacteriophage strain with the pharmaceutically acceptable excipient when infection or lysis of the pathogen and the second pathogen are detected.

In various embodiments, the composition includes an amount of a third bacteriophage strain effective for reducing a concentration of a third pathogen of a species different from the species of the pathogen and second pathogen and in the intestinal microbiome of the subject, wherein the third pathogen induces obesity, inflammation, or an obesity-associated metabolic disorder and the third bacteriophage strain has an infectivity specific to a species of the third pathogen. In various embodiments are disclosed processes of preparing compositions of various embodiments including: characterizing the bacteriophage strain by exposing the pathogen to the bacteriophage strain and detecting infection or lysis of the pathogen; characterizing the second bacteriophage strain by exposing the second pathogen to the second bacteriophage strain and detecting infection or lysis of the second pathogen; characterizing the third bacteriophage strain by exposing the third pathogen to the third bacteriophage strain and detecting infection or lysis of the third pathogen; and combining the bacteriophage strain, the second bacteriophage strain, and third bacteriophage strain with the pharmaceutically acceptable excipient when infection or lysis of the pathogen, the second pathogen, and the third pathogen are detected.

In various embodiments, the composition includes an amount of a fourth bacteriophage strain effective for reducing a concentration of a fourth pathogen of a species different from the species of the pathogen, second pathogen, and third pathogen and in the intestinal microbiome of the subject, wherein the fourth pathogen induces obesity, inflammation, or an obesity-associated metabolic disorder and the fourth bacteriophage strain has an infectivity specific to a species of the fourth pathogen. As previously highlighted, the fourth pathogen is understood to mean four or more different pathogens. In various embodiments are disclosed processes of preparing compositions of various embodiments including: characterizing the bacteriophage strain by exposing the pathogen to the bacteriophage strain and detecting infection or lysis of the pathogen; characterizing the second bacteriophage strain by exposing the second pathogen to the second bacteriophage strain and detecting infection or lysis of the second pathogen; characterizing the third bacteriophage strain by exposing the third pathogen to the third bacteriophage strain and detecting infection or lysis of the third pathogen; characterizing the fourth bacteriophage strain by exposing the fourth pathogen to the fourth bacteriophage strain and detecting infection or lysis of the fourth pathogen; and combining the bacteriophage strain, the second bacteriophage strain, third bacteriophage strain, and fourth bacteriophage strain with the pharmaceutically acceptable excipient when infection or lysis of the pathogen, the second pathogen, the third pathogen, and the fourth pathogen are detected.

In various embodiments, the pharmaceutically acceptable excipient is a carrier suitable for oral consumption. Examples of carriers include silicon dioxide (silica, silica gel), carbohydrates or carbohydrate polymers (polysaccharides), cyclodextrins, starches, degraded starches (starch hydrolysates), chemically or physically modified starches, modified celluloses, gum arabic, ghatti gum, tragacanth, karaya, carrageenan, guar gum, locust bean gum, alginates, pectin, inulin or xanthan gum, or hydrolysates of maltodextrins and dextrins. In various embodiments, the bacteriophage strain is dispersed throughout the carrier.

In various embodiments, the pharmaceutically acceptable excipient is capable of delivering at least a portion of the amount of the bacteriophage strain to the intestinal microbiome of the subject in an active state such that the at least a portion of the amount of the bacteriophage strain is capable of infecting and lysing the pathogen.

In various embodiments, the pharmaceutically acceptable excipient includes an extended release phase capable of releasing at least a portion of the amount of the bacteriophage strain to the intestinal microbiome of the subject over a period of time. In other embodiments, the pharmaceutically acceptable excipient includes an immediate release phase capable of substantially immediately releasing at least a portion of the amount of the bacteriophage strain to the intestinal microbiome of the subject.

In various embodiments, the composition includes a probiotic microorganism capable of colonizing the intestinal microbiome of the subject or a substance capable of stimulating growth of microorganisms with beneficial properties. Examples of probiotic microorganisms include: Lactobacillus such as L. plantarum, L. paracasei, L. acidophilus, L. casei, L. rhamnosus, L. crispatus, L. gasseri, L. reuteri, L. bulgaricus; Bifidobacterium such as B. longum, B. catenulatum, B. breve, B. animalis, B. bifidum; Streptococcus such as S. sanguis, S. oralis, S. mitis, S. thermophilus, S. salivarius; Bacillus such as B. coagulans, B. subtilis, B. laterosporus; Lactococcus such as L. lactis; Enterococcus such as E. faecium; Pediococcus such as P. acidilactici; Propionibacterium suchas P. jensenii, P. freudenreichii; Peptostreptococcus such as P. productus; and Saccharomyces such as S. boulardii.

In various embodiments are disclosed methods for altering an intestinal microbiome of a subject to treat obesity, inflammation, or obesity-associated metabolic disorders including: administering an amount of a bacteriophage strain to a subject with an intestinal microbiome having a concentration of a pathogen inducing obesity, inflammation, or an obesity-associated metabolic disorder, wherein the amount of a bacteriophage strain reduces the concentration of the pathogen and the bacteriophage strain has an infectivity specific to a species of the pathogen. The bacteriophage strain of various embodiments is at least two different bacteriophage strains, where each bacteriophage strain has an infectivity specific to the species of the pathogen.

The amount of the bacteriophage strain of various embodiments when administered reduces the concentration of the pathogen by or at least by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In various embodiments, the amount of the bacteriophage strain reduces the concentration of the pathogen between any two percentages from above.

The amount of the bacteriophage strain of various embodiments when administered reduces the inflammation by or at least by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In various embodiments, the amount of the bacteriophage strain reduces the inflammation in the subject between any two percentages from above. The reduction of inflammation includes, for example, reductions in various inflammatory factors such as, for example, cytokines, chemokines, eicosanoids, etc.

The amount of bacteriophage strain of various embodiments when administered preferably reduces the weight of the subject by or at least by 11%, 2%, 3%, 4%, 5%, 6%, 7%, 8%, 9%, 10%, 11%, 12%, 13%, 14%, 15%, 16%, 17%, 18%, 19%, 2, 2 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In various embodiments, the amount of the bacteriophage strain reduces the weight of the subject between any two percentages from above.

In various embodiments, the administering of the bacteriophage strain is used to treat, for example, chronic illnesses such as diabetes, heart attacks, strokes, and cancers.

In various embodiments, the administering of the bacteriophage strain is used to treat obesity-associated metabolic disorders including, for example, insulin insensitivity, type 2 diabetes, hepatic steatosis (fatty liver disease), atherosclerosis, heart disease, and cancers.

In various embodiments, the administering includes administering an amount of a second bacteriophage strain to the subject with the intestinal microbiome having a concentration of a second pathogen of a species different from the species of the pathogen and inducing obesity, inflammation, or an obesity-associated metabolic disorder, wherein the amount of the second bacteriophage strain reduces the concentration of the second pathogen and the second bacteriophage strain has an infectivity specific to the species of the second pathogen. An amount of a second bacteriophage strain is also understood to mean compositions of various embodiments having amounts of multiple bacteriophage strains with each of the multiple bacteriophage strains having infectivity specific to different pathogens and species of the different pathogens.

In various embodiments, the administering includes administering an amount of a third bacteriophage strain to the subject with the intestinal microbiome having a concentration of a third pathogen of a species different from the species of the pathogen and the second pathogen and inducing obesity, inflammation, or an obesity-associated metabolic disorder, wherein the amount of the third bacteriophage strain reduces the concentration of the third pathogen and the third bacteriophage strain has an infectivity specific to the species of the third pathogen.

In various embodiments, the administering includes administering an amount of a fourth bacteriophage strain to the subject with the intestinal microbiome having a concentration of a fourth pathogen of a species different from the species of the pathogen, the second pathogen, and the third pathogen and inducing obesity, inflammation, or an obesity-associated metabolic disorder, wherein the amount of the fourth bacteriophage strain reduces the concentration of the fourth pathogen and the fourth bacteriophage strain has an infectivity specific to the species of the fourth pathogen. As previously highlighted, the fourth pathogen is understood to mean four or more different pathogens.

In various embodiments are disclosed processes for preparing a composition for altering an intestinal microbiome of a subject to treat obesity, inflammation, or obesity-associated metabolic disorders, the processes including: identifying a pathogen, where a concentration of the pathogen in the intestinal microbiome of the subject induces obesity, inflammation, or an obesity-associated metabolic disorder; characterizing a bacteriophage strain by exposing the pathogen to the bacteriophage strain and detecting infection or lysis of the pathogen; and preparing a composition for altering the intestinal microbiome of a subject to treat obesity, inflammation, or an obesity-associated metabolic disorder by combining the bacteriophage strain with a pharmaceutically acceptable excipient when infection or lysis of the pathogen is detected. In various embodiments, the bacteriophage strain is different bacteriophage strains, the characterizing includes characterizing each bacteriophage strain by separately exposing the pathogen to each bacteriophage strain and detecting infection or lysis of the pathogen, and the preparing includes combining at least two different bacteriophage strains that infect or lyse the pathogen with the pharmaceutically acceptable excipient. The processes of various embodiments include isolating the pathogen(s) or the bacteriophage strain(s).

In various embodiments, the characterizing includes exposing a second pathogen from a species of the pathogen with the bacteriophage strain and detecting infection or lysis of the second pathogen and the combining includes combining the bacteriophage strain with the pharmaceutically acceptable excipient when infection or lysis of the second pathogen is detected. In other embodiments, the characterizing includes exposing at least a second pathogen from a species different from the pathogen with the bacteriophage strain and detecting infection or lysis of the second pathogen and the combining includes combining the bacteriophage strain with the pharmaceutically acceptable excipient when infection or lysis of the second pathogen by the bacteriophage strain is not detected. For example, the bacteriophage strain is characterized using a pathogen and a beneficial microorganism. Further, the second pathogen is understood to be at least two different pathogens.

The processes of various embodiments includes: identifying a second pathogen of a species different from the pathogen, where a concentration of the second pathogen in the intestinal microbiome of the subject induces obesity, inflammation, or an obesity-associated metabolic disorder; and characterizing a second bacteriophage strain by exposing the second pathogen to the second bacteriophage strain and detecting infection or lysis of the second pathogen; wherein the preparing includes combining the second bacteriophage strain with the bacteriophage strain and the pharmaceutically acceptable excipient when infection or lysis of the second pathogen is detected. A second bacteriophage strain of various embodiments is also understood to mean multiple bacteriophage strains with each of the multiple bacteriophage strains having infectivity specific to different pathogens and species of the different pathogens. In various embodiments, the second bacteriophage strain is different second bacteriophage strains, the characterizing of the second bacteriophage strain includes characterizing each second bacteriophage strain by separately exposing the second pathogen to each second bacteriophage strain and detecting infection or lysis of the second pathogen, and the preparing includes combining at least two different second bacteriophage strains that infect or lyse the second pathogen with the bacteriophage strain and the pharmaceutically acceptable excipient.

The processes of various embodiments includes: identifying a third pathogen of a species different from the pathogen and the second pathogen, where a concentration of the third pathogen in the intestinal microbiome of the subject induces obesity, inflammation, or an obesity-associated metabolic disorder; and characterizing a third bacteriophage strain by exposing the third pathogen to the third bacteriophage strain and detecting infection or lysis of the third pathogen; wherein the preparing includes combining the third bacteriophage strain with the second bacteriophage strain, bacteriophage strain, and the pharmaceutically acceptable excipient when infection or lysis of the third pathogen is detected. In various embodiments, the third bacteriophage strain is different third bacteriophage strains, the characterizing of the third bacteriophage strain includes characterizing each third bacteriophage strain by separately exposing the third pathogen to each third bacteriophage strain and detecting infection or lysis of the third pathogen, and the preparing includes combining at least two different third bacteriophage strains that infect or lyse the third pathogen with the second bacteriophage strain, bacteriophage strain, and the pharmaceutically acceptable excipient.

The processes of various embodiments includes: identifying a fourth pathogen of a species different from the pathogen, the second pathogen, and the third pathogen, where a concentration of the fourth pathogen in the intestinal microbiome of the subject induces obesity, inflammation, or an obesity-associated metabolic disorder; and characterizing a fourth bacteriophage strain by exposing the fourth pathogen to the fourth bacteriophage strain and detecting infection or lysis of the fourth pathogen; wherein the preparing includes combining the fourth bacteriophage strain with the third bacteriophage strain, the second bacteriophage strain, bacteriophage strain, and the pharmaceutically acceptable excipient when infection or lysis of the fourth pathogen is detected. As previously highlighted, the fourth pathogen is understood to mean four or more different pathogens. In various embodiments, the fourth bacteriophage strain is different fourth bacteriophage strains, the characterizing of the fourth bacteriophage strain includes characterizing each fourth bacteriophage strain by separately exposing the fourth pathogen to each fourth bacteriophage strain and detecting infection or lysis of the fourth pathogen, and the preparing includes combining at least two different fourth bacteriophage strains that infect or lyse the fourth pathogen with the third bacteriophage strain, the second bacteriophage strain, the bacteriophage strain, and the pharmaceutically acceptable excipient.

The following examples illustrate the various embodiments of the present invention. Those skilled in the art will recognize many variations that are within the spirit of the present invention and scope of the claims.

### Example 1

### Lytic Activity Assay

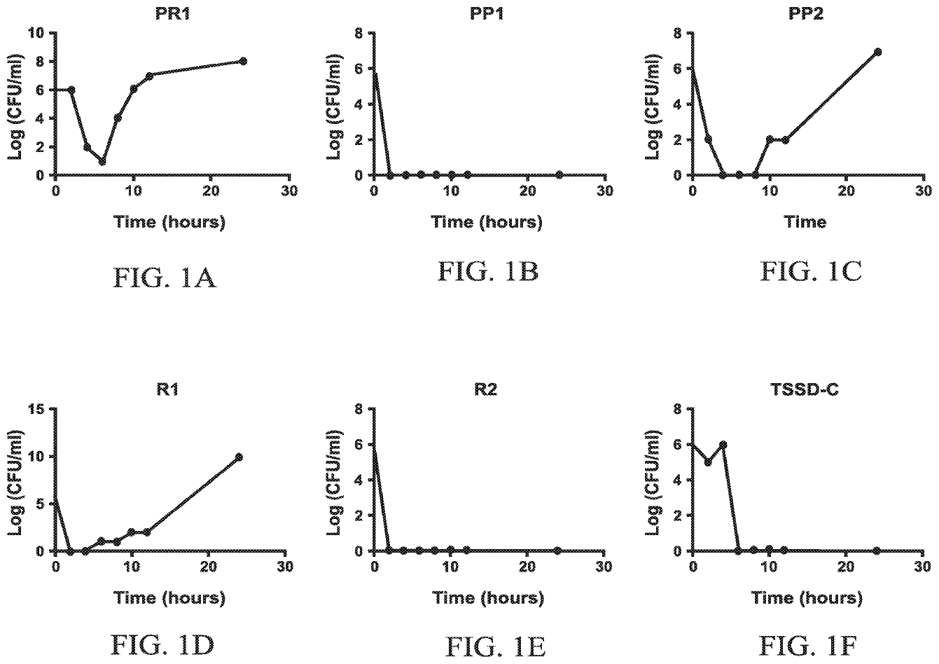

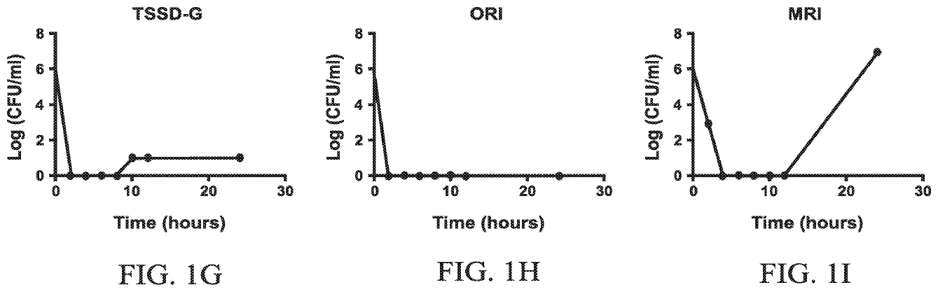

Thirteen phages are isolated from local sewage plants in Salt Lake and Utah counties. Enterobacter cloacae strain B29 is also acquired. These strains are analyzed by using a plaque assay to infect Enterobacter cloacae strain B29. FIGS. 1A, 1B, 1C, 1D, 1E, 1F, 1G, 1H, and 1I are graphical representations showing lytic activity of bacteriophage strains (PR1, PP1, PP2, R1, R2, TSSD-C, TSSD-G, ORI, and MRI) on Enterobacter cloacae strain B29. As shown in FIG. 1A-1I, the lytic activity assays performed with an MOI of 100 show that many of the bacteriophage strains are strongly lytic to Enterobacter cloacae strain B29 and can be used to kill Enterobacter cloacae bacteria in the gut.

### Example 2

### Phage Isolation

Environmental samples are collected from sewage treatment facilities and enriched by growing with Enterobacter cloacae strain B29 for two days. Cultures are then centrifuged and filtered to remove debris and bacteria and to isolate phage. The filtrate is passed through three rounds of purification by plaque forming assay. Following purification, phage genomic DNA is isolated and sequenced, and genomes are assembled bioinformatically. Phages are determined to be novel by using BLAST search comparisons to existing databases, and all are found to have no more than 99% identity to the closest related phage in the database. GEPARD dotplotter is used to compare genome relatedness two-by-two, and it is verified that these phages are not identical to each other or to any in the existing genomic databases. The genomes of all lytic phage identified are thoroughly annotated, with particular attention to identifying any toxin genes that may be present in the phage genomes. Only phages not containing toxin genes are considered for subsequent development of a therapeutic cocktail.

### Gut Survival Ability Testing

Bacteriophage (or phage) are viruses that can infect and lyse bacteria but not other kinds of cells. Because they have no ability to infect human cells, they are being considered as a potential therapeutic alternative to antibiotics to treat specific bacterial infections and dysbiosis of the gut microbiota in humans. Dysbiosis of the gut microbiota has recently been shown to play an important role in the development of obesity. One goal is to isolate phages that have specific and targeted lytic activity against a bacterial species (Enterobacter cloacae strain B29) that has been shown to cause obesity in a human and a mouse model, in order to eventually develop a phage cocktail that can suppress growth of this bacteria in the gut. From environmental samples, we isolate several phage strains with lytic activity against this bacteria in plaque-forming assays. The effort to isolate more phage representing a broad range of phage families and lytic activities is ongoing. In order for these phages to be considered as potential therapeutics, however, we demonstrate that they can survive conditions in the gastrointestinal tract. The purpose of this protocol is to measure phage survival ability of our various phage isolates by administering solutions of phage to mice by oral gavage, then collecting fecal samples to measure phage concentration. Phages shown by these tests to survive the digestive tract are considered for subsequent development of a therapeutic cocktail.

For each phage to be tested, male C57 mice (6 weeks old) are purchased and divided into negative control (n=3) and test groups (n=7). The control group are given sterile Luria broth (LB) (0.2 ml) via oral-gavage once. Test group are given 10¹⁰ pfu/ml of phage in 0.2 ml of LB broth via oral-gavage once. This dose of phage is selected based on a prior publication by Majewska²¹, which is incorporated by reference, that measured survival of different phage in the C57/B16 mouse digestive tract, which demonstrates that a dose of 2×10¹⁰ phage is more than sufficient to detect surviving phage by standard plaque assay. Phages are grown in and purified from host bacteria, and titer is determined by standard plaque assay before diluting the phage to the stated concentration in Luria broth. Immediately before the oral gavage, fecal samples are collected from all mice. Fecal samples are also collected from all mice every 6 hours until the 24th hour after gavage. Immediately after collection, each fecal sample is resuspended in phosphate buffered saline and filtered through a 0.45 um filter to isolate phage. The filtered solution is grown with host bacteria in a standard plaque-forming assay to calculate phage concentration in the fecal sample and thus determine how well the phage have survived the digestive tract. Table 1 shows survival ability of 9 Enterobacter cloacae strain B29 phages after passage through the mouse gastrointestinal tract. For example as shown in Table 1, the different phages are capable of surviving within the digestive tract for an extended period of time.

TABLE 1

Gut Survivalbility Test: Time (hrs)

| Phages: | 0 | 6 | 12 | 24 |
|---|---|---|---|---|
| MRI | − | + | + | + |
| PRI | − | + | + | + |
| PP1 | − | + | + | + |
| R1 | − | + | + | + |
| ORI | − | + | + | + |
| TSSD-C | − | + | + | + |
| TSSD-G | − | + | + | + |
| PP2 | − | + | + | + |
| R2 | − | + | + | + |

### Lytic Activity Assay

Temperate phages undergo lysogeny and integrate into the host genome. Lytic phages, in contrast, replicate in and then burst the host cell, destroying it. Lytic phages are needed for our purposes. To identify lytic phages, Enterobacter cloacae strain B29 is diluted from an over-night culture to a concentration of 10⁶ cfu/ml and inoculated with phage at a concentration of 10⁸ pfu/ml, giving a multiplicity of infection (MOI) of 100. One ml of sample is removed from the flask, serial diluted immediately, and spread over LB-agar for overnight culture. The amount of surviving bacteria is determined by colony count the next day. Additional 1 ml samples are removed from the culture every 2 hours during the first 12 hours after inoculation then once more at the 24^{th} hour after inoculation, to detect whether the phage is lytic and whether bacteria develop resistance within the first 24 hours (FIG. 1).

### Host Range Testing

The target specificity of each phage strain is tested by measuring its ability to lyse a range of bacteria, from closely to distantly related bacterial strains. For example, Tables 2 and 3 illustrates host range testing of 9 phage strains that were initially isolated based on their ability to lyse Enterobacter cloacae strain B29. Each phage was tested against two other E. cloacae strains, ATCC 13047 and 23855, as well as more distantly related Shigella boydii Ewing, Klebsiella pneumoniae, Salmonella typhimurium, Escherichia coli, Serratia marcescens, and Pseudomonas aeruginosa. A spot test is performed by dropping 100 μl of a phage solution containing 10⁹ pfu/ml onto a lawn of bacteria and incubating overnight at 37° C. Tables 2 and 3 show host range testing of 9 Enterobacter cloacae strain B29 phages using spot-testing, where clearing of bacteria in a spot indicates the lytic activity of a phage in a bacterial strain. For example as shown in Tables 2 and 3, the different phages display infectivities specific to different strains of Enterobacter cloacae and are capable of lysing the different strains of Enterobacter cloacae.

TABLE 2

| | Spot test | | | | |
|---|---|---|---|---|---|
| Species | MR1 | PR1 | PP1 | R1 | ORI |
| E. cloacae B29 | + | + | + | + | + |
| E. cloacae ATCC 13047 | + | + | + | + | + |
| E. cloacae ATCC 23855 | + | + | + | + | + |
| Shigella ATCC 9207 | − | − | − | − | − |
| klebsiella ATCC 10031 | − | − | − | − | − |
| Salmonella LT2 | − | − | − | − | − |
| E. coli 814 | − | − | − | − | − |
| S. marcescens | − | − | − | − | − |
| P. aeruginosa | + | − | − | − | − |

TABLE 3

| | Spot test | | | |
|---|---|---|---|---|
| Species | TSSD-C | TSSD-G | PP2 | R2 |
| E. cloacae B29 | + | + | + | + |
| E. cloacae ATCC 13047 | + | + | + | + |
| E. cloacae ATCC 23855 | + | + | − | + |
| Shigella ATCC 9207 | − | − | − | − |
| klebsiella ATCC 10031 | − | − | − | − |
| Salmonella LT2 | − | − | − | − |
| E. coli 814 | − | − | − | − |
| S. marcescens | − | − | − | − |
| P. aeruginosa | − | − | − | − |

The lytic activity of the phages toward each vulnerable strain of bacteria is further quantified by mixing 100 μl of 10⁹ pfu/ml phage with 500 μl 10⁹ cfu/ml bacteria and incubating overnight at 37° C. with shaking, then performing serial dilution plating the next day and counting plaques to assess each phage's ability to replicate in the host bacteria (Tables 4 and 5). Tables 4 and 5 show host range testing of 9 Enterobacter cloacae strain B29 using bacterial infection followed by plaque assay. For example as shown in Tables 4 and 5, the different phages display infectivities specific to different strains of Enterobacter cloacae and are capable of replicating within and lysing the different strains of Enterobacter cloacae.

TABLE 4

| | Efficiency of plating (PFU/ml) | | | | |
|---|---|---|---|---|---|
| Species | MR1 | PR1 | PP1 | R1 | ORI |
| E. cloacae B29 | 1.77 × 10\hat{ }11 | 3.88 × 10\hat{ }11 | 2.31 × 10\hat{ }10 | 3.31 × 10\hat{ }11 | 2.87 × 10\hat{ }8 |
| E. cloacae ATCC 13047 | 3.78 × 10\hat{ }10 | 2.12 × 10\hat{ }10 | 5.55 × 10\hat{ }7 | 1.12 × 10\hat{ }13 | 4.68 × 10\hat{ }11 |
| E. cloacae ATCC 23855 | 1.33 × 10\hat{ }7 | 1.44 × 10\hat{ }8 | 1.722 × 10\hat{ }4 | 8.22 × 10\hat{ }5 | 3.33 × 10\hat{ }8 |
| Shigella ATCC 9207 | ND | ND | ND | ND | ND |
| klebsiella ATCC 10031 | ND | ND | ND | ND | ND |
| Salmonella LT2 | ND | ND | ND | ND | ND |
| E. coli 814 | ND | ND | ND | ND | ND |
| S. marcescens | ND | ND | ND | ND | ND |
| P. aeruginosa | 1.38 × 10\hat{ }7 | ND | ND | ND | ND |

TABLE 5

| | Efficiency of plating (PFU/ml) | | | |
|---|---|---|---|---|
| Species | TSSD-C | TSSD-G | PP2 | R2 |
| E. cloacae B29 | 5.55 × 10\hat{ }7 | 6.13 × 10\hat{ }8 | 7.46 × 10\hat{ }10 | 1.27 × 10\hat{ }11 |
| E. cloacae ATCC 13047 | 4.81 × 10\hat{ }8 | 1.16 × 10\hat{ }9 | 1.68 × 10\hat{ }11 | 1.58 × 10\hat{ }13 |
| E. cloacae ATCC 23855 | 2.22 × 10\hat{ }5 | 2.44 × 10\hat{ }7 | ND | 5.01 × 10\hat{ }8 |
| Shigella ATCC 9207 | ND | ND | ND | ND |
| klebsiella ATCC 10031 | ND | ND | ND | ND |
| Salmonella LT2 | ND | ND | ND | ND |
| E. coli 814 | ND | ND | ND | ND |
| S. marcescens | ND | ND | ND | ND |
| P. aeruginosa | ND | ND | ND | ND |

### Animal Testing

In vivo proof-of-principle testing are performed to determine whether phage treatment of gut Enterobacter cloacae strain B29 can reduce inflammation, obesity, or associated metabolic disorders in an animal model. Germ-free mice are inoculated orally with Enterobacter cloacae strain B29, which is shown to cause inflammation and obesity in the mice. Mice are treated by oral administration of phage that are found to be lytic and to not contain toxin genes. Phages are tested individually and in combinations of two or more phages, to determine whether inflammation or obesity decreases and to guide formulation of a cocktail to which bacteria are unlikely to develop resistance because it contains multiple phages that are not closely related to each other.

### Example 3

Enterobacter cloacae strain B29 is selected as the obesogenic gut microbe on which to perform proof-of-concept studies. An Enterobacter cloacae strain B29-induced obesity model upon which phage can be tested is established in mice, using broad-spectrum antibiotics to first deplete the native mouse microbiota so that Enterobacter cloacae strain B29, which does not normally colonize mice, engrafts. Nine Enterobacter cloacae strain B29-specific phages are isolated, verified as lytic, and shown to survive conditions in the mammalian gastrointestinal (GI) tract. Multiple phages that attack their target through different surface receptors are used so bacteria are unlikely to evolve resistance.

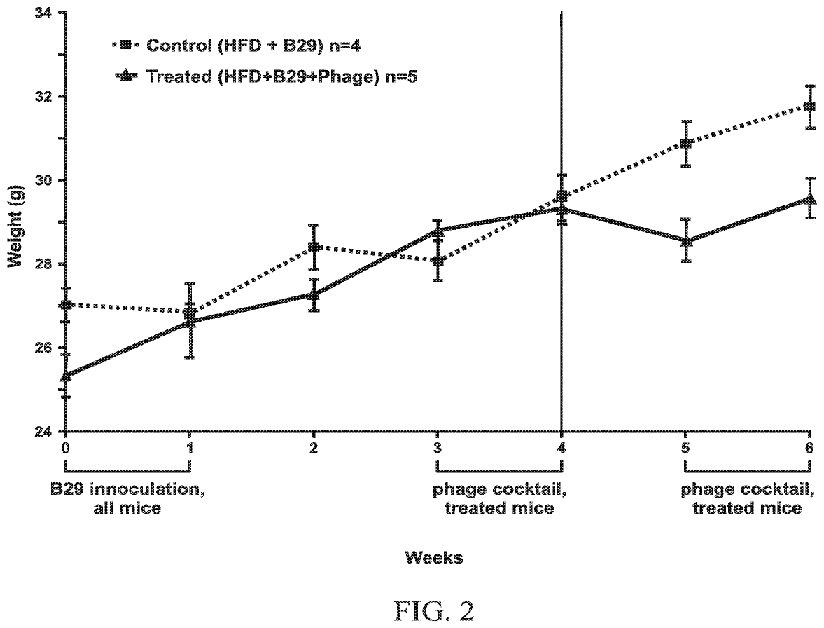

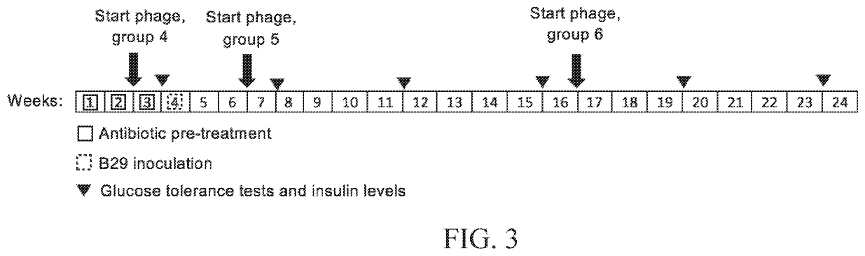

FIG. 2 is a graphical representation showing arrest of weight gain in mice with intestinal microbiomes colonized with Enterobacter cloacae strain B29 treated with bacteriophages as compared to control mice with intestinal microbiomes colonized with Enterobacter cloacae strain B29. As shown in FIG. 2, phage cocktail inhibits B29-induced weight gain. Mice are treated with oral antibiotics to deplete their native gut microbiota. During week 1, all mice receive daily doses of B29 by oral gavage and are placed on a high fat diet to induce weigh gain. At week 3, the treatment group (black line) receives 7 daily doses of phage cocktail by oral gavage. The phage treatment is repeated at week 5. Body weight is monitored weekly throughout the study and is significantly different in phage-treated group as compared to control group at weeks 5 and 6 (*, p<0.05).

### Live Animal Study

The study includes 6 groups of 8 mice each. The study groups include:

Group 1: Obese control—normal gut microbiota; develops obesity.
Group 2: Lean control—antibiotic-depleted gut microbiota; does not develop obesity since gut microbiota is required.
Group 3: Obese control—antibiotic-deplete then inoculate with B29 to reconstitute gut microbiota; develops obesity.
Group 4: Experimental—administer phage cocktail at beginning of week 3 during antibiotic-depletion, one week prior to first inoculation with B29; phage prevents engraftment of B29 and prevents obesity development.
Group 5: Experimental—administer phage cocktail at the beginning of week 7 after B29 has engrafted but before obesity develops; phage prevents development of obesity by reducing B29 levels and inflammation.
Group 6: Experimental—administer phage cocktail at the beginning of week 17 after obesity has developed; phage decreases systemic inflammation and induces weight loss by reducing B29 levels.

FIG. 3 is a graphical representation showing a study designed to measure the efficacy of phage treatment when administered before exposure to Enterobacter cloacae strain B29, after exposure to Enterobacter cloacae strain B29 but before obesity develops, and after obesity develops due to colonization of the gut microbiota by Enterobacter cloacae strain B29. Broad-spectrum antibiotics are administered to Groups 2-6 (see group descriptions above) to deplete their native gut microbiota during weeks 1-3, then their microbiotas are reconstituted (except group 2) by twice-daily oral gavage with 10¹⁰ colony forming units of Enterobacter cloacae strain B29 during week 4. All mice are fed a high fat diet (HFD) at the start of week 5 and remain on it for the duration of the study. The methods for antibiotic depletion and Enterobacter cloacae strain B29 reconstitution of the gut microbiota are tested and prove effective. When the Enterobacter cloacae strain B29 levels decrease in group 3 controls during the study, a biweekly “booster” doses of Enterobacter cloacae strain B29 is administered to groups 3-6. Bodyweight is measured weekly throughout the study. Phage treatments are initiated at the time points indicated in FIG. 3 and administered by daily oral gavage for the first week, then by inclusion in drinking water for the duration of the study. Fecal samples are collected and cultured biweekly beginning at the end of week 4 to track Enterobacter cloacae strain B29 levels. Glucose tolerance and insulin are measured every 4 weeks as shown below, to detect type 2 diabetes. By 12 weeks, we demonstrate obesity in control mice. By 24 weeks, conclusions regarding the effect of the Enterobacter cloacae strain B29 phage cocktail on body weight and glucose tolerance when given before Enterobacter cloacae strain B29 exposure, after Enterobacter cloacae strain B29 engraftment but before obesity develops, or after Enterobacter cloacae strain B29 engraftment and after obesity is established.

### Molecular Level Analyses of Mouse Tissues for Markers of Inflammation and Insulin Resistance

Each mouse is dissected at the end of the study and various tissues are preserved. Molecular markers of inflammation such as tumor necrosis factor α(TNFα), interleukin 1β(IL-1β), interleukin (IL-6), toll like receptor 4 (Tlr4), and IkappaB kinase epsilon (Ikkε) are examined in liver, intestine, and adipose tissue using qRT-PCR. ELISA assays are used to measure serum levels of proteins that influence blood sugar and appetite (insuin, leptin, and adiponectin), and inflammation (serum amyloid A and LPS-binding protein). Some or all molecular markers of inflammation and glucose dysregulation improve in response to the administration of the phage cocktail. Additional markers of insulin activity (AccI, Fas, Fiaf, Srebpl, Pparq) and gut permeability (Zol, Occludin, Claudin) are examined at the RNA and protein levels in tissues. The mechanisms underlying bodyweight changes are deduced by quantification of the molecular-level changes occurring in mice in response to the phage cocktail.

### Isolation of Phages Against Two More Obesogenic Human Gut Bacteria

To expand the range of people who can potentially benefit from the phage cocktail, phages against three more commonly occurring obesogenic gut microbes are isolated and characterized for inclusion in the cocktail. Bacterial candidates include Clostridium ramosum, which is found to be elevated in patients with obesity and type 2 diabetes and caused obesity when transplanted into germ-free mice¹⁷⁻¹⁹, as well as two other bacterial strains. Phages are isolated and characterized using the same standard techniques as for the Enterobacter cloacae strain B29 phages. The lytic activity and host range of each are tested in vitro, and only lytic phages with high host specificity remain as candidates for the cocktail. Finally, the ability of each phage to survive conditions in the mammalian GI tract is tested in mice. Thus, 10-20 additional phages specific for three more obesogenic gut bacteria are each validated as lytic, with a narrow host range, and able to survive the mammalian GI tract.

### Genome Sequencing and Annotation of all Phages Intended for the Cocktail

Phages cannot infect human or other eukaryotic cell types, and their narrow host range ensures that they will not infect beneficial gut microbes, so there is no risk of off-target infection by the phage cocktail. Some phages, however, have acquired bacterial toxin genes at some point in their evolutionary history. To ensure that no phages carrying bacterial toxin genes are included in the phage cocktail, complete genome sequencing of every phage is performed. A phage strain found to contain toxin genes is removed from further consideration. In the unlikely case that toxin genes occur more frequently than expected and fewer than three phages per bacterial target are left for inclusion in the cocktail, additional phages are isolated and characterized using the same techniques as described above. Thus, the final formulation of the phage cocktail, including approximately 3-6 fully characterized and safety validated phages against each of three different obesogenic human gut bacterial species, for a total of approximately 9-18 phages is provided.

### Commercialization and Economic Impact

The phage cocktail works in combination with probiotic supplements, eliminating select harmful gut microbes to create ecological space for probiotics to thrive. The probiotic market is therefore the best indicator of the phage cocktail's potential economic impact. The global market for probiotic supplements was $3.3 billion in 2015 and is projected to continue growing at 7.6% per year until 2025²². It is the fastest-growing segment of the nutritional supplements market. In dietary supplements overall, supplements targeted at weight loss, gastrointestinal health, and diabetes constitute 3 of the top 5 fastest growing sales categories²³.

The present disclosure as disclosed in various embodiments can be scaled-up and optimizing a delivery system. Examples of pharmaceutical compositions including the composition of any embodiments can be in the form of softgels or liquid, or freeze-dried phages in capsules, powder, or effervescent tablets. Enteric coatings are possible but probably not necessary—passage of naked Enterobacter cloacae strain B29 phages through the mouse stomach resulted in little or no diminution of phage viability. From then on, new phages can be added to the cocktail as additional obesogenic bacteria are discovered.

### REFERENCES

1. Fei N & Zhao L (2013) An opportunistic pathogen isolated from the gut of an obese human causes obesity in germfree mice. ISME J 7: 880-884.
2. Flegal K M, Kruszon-Moran D, Carroll M D, Fryar C D, Ogden C L. (2016) Trends in obesity among adults in the United States, 2005 to 2014. Journal of the American Medical Association. 315(21):2284-91.
3. IARC (International Agency for Research on Cancer). 2002. Weight Control and Physical Activity. Lyon, France: IARC Press.
4. Li Q, Blume S W, Huang J C, Hammer M, Ganz M L: Prevalence and healthcare costs of obesity-related comorbidities: evidence from an electronic medical records system in the United States. Journal of medical economics 2015, 18(12):1020-1028.
5. Li Q, Blume S W, Huang J C, Hammer M, Graf T R: The Economic Burden of Obesity by Glycemic Stage in the United States. PharmacoEconomics 2015, 33(7):735-748.
6. Majewska, J., et al. (2015) “Oral application of T4 phage induces weak antibody production in the gut and in the blood.” Viruses 7:4783-4799.
7. Ogden C L, Carroll M D, Kit B K, Flegal K M. (2012) Prevalence of obesity and trends in body mass index among US children and adolescents, 1999-2010. Journal of the American Medical Association. 307(5):483-90.
8. Penadés, José R., et al. (2015) “Bacteriophage-mediated spread of bacterial virulence genes.” Current opinion in microbiology 23: 171-178.
9. Sturm R: Increases in clinically severe obesity in the United States, 1986-2000. Archives of internal medicine 2003, 163(18):2146-2148.
10. Sturm R, Hattori A: Morbid Obesity Rates Continue to Rise Rapidly in the US. International journal of obesity 2013, 37(6):889-891.
11. Sonnenburg J L, Backhed F: Diet-microbiota interactions as moderators of human metabolism. Nature 2016, 535(7610):56-64.
12. Ridaura V K, Faith J J, Rey F E, Cheng J, Duncan A E, Kau A L: Gut microbiota from twins discordant for obesity modulate metabolism in mice. Science 2013, 341.
13. Shen J, Obin M S, Zhao L: The gut microbiota, obesity and insulin resistance. Molecular aspects of medicine 2013, 34(1):39-58.
14. Murphy E A, Velazquez K T, Herbert K M: Influence of high-fat diet on gut microbiota: a driving force for chronic disease risk. Current opinion in clinical nutrition and metabolic care 2015.
15. Cani P D, Amar J, Iglesias M A, Poggi M, Knauf C, Bastelica D: Metabolic endotoxemia initiates obesity and insulin resistance. Diabetes 2007, 56.
16. Ferrer M, Ruiz A, Lanza F, Haange S B, Oberbach A, Till H, Bargiela R, Campoy C, Segura M T, Richter M et al: Microbiota from the distal guts of lean and obese adolescents exhibit partial functional redundancy besides clear differences in community structure. Environ Microbiol 2013, 15(1):211-226.
17. Le Chatelier E, Nielsen T, Qin J, Prifti E, Hildebrand F, Falony G, Almeida M, Arumugam M, Batto J M, Kennedy S et al: Richness of human gut microbiome correlates with metabolic markers. Nature 2013, 500(7464):541-546.
18. Karlsson F H, Tremaroli V, Nookaew I, Bergstrom G, Behre C J, Fagerberg B, Nielsen J, Backhed F: Gut metagenome in European women with normal, impaired and diabetic glucose control. Nature 2013, 498(7452):99-103.
19. Woting A, Pfeiffer N, Loh G, Klaus S, Blaut M: Clostridium ramosum promotes high-fat diet-induced obesity in gnotobiotic mouse models. MBio 2014, 5(5):e01530-01514.
20. Thaiss C A, Itav S, Rothschild D, Meijer M, Levy M, Moresi C, Dohnalova L, Braverman S, Rozin S, Malitsky S et al: Persistent microbiome alterations modulate the rate of post-dieting weight regain. Nature 2016.
21. Majewska, Joanna, et al. “Oral application of T4 phage induces weak antibody production in the gut and in the blood.” Viruses 7.8 (2015): 4783-4799.
22. Probiotics Dietary Supplements Market Analysis by Application (Food Supplements, Nutritional Supplements, Specialty Nutrients, Infant Formula), By Regions (North America, Europe, Asia Pacific, Middle East & Africa, CSA), And Segment Forecasts, 2014-2025. In.: Grand View Research; 2017.
23. 20th Annual Nutrition Business Journal Summit (http://www.newhope.com/sites/newhope360.com/files/Expo-West-2017-Supplements-Today-1.pdf, accessed on 14 Mar. 2018).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A composition for altering an intestinal microbiome of a subject to treat obesity, inflammation, or obesity-associated metabolic disorders, the composition comprising an amount of at least one bacteriophage strain effective for reducing a concentration of a pathogen in an intestinal microbiome of a subject and a pharmaceutically acceptable excipient, wherein the concentration of the pathogen induces obesity, inflammation, or an obesity-associated metabolic disorder and the bacteriophage strain has an infectivity specific to a species of the pathogen.

2. The composition of claim 1, wherein the pathogen belongs to a genus Enterobacter.

3. The composition of claim 1, wherein the pathogen is Enterobacter cloacae strain B29.

4. (canceled)

5. The composition of claim 1, wherein the bacteriophage strain is adapted to reduce the concentration of the pathogen by infecting and causing lysis of the pathogen.

6. The composition of claim 5, wherein the bacteriophage strain is adapted to infect a binding site or surface receptor unique to the pathogen or the species of the pathogen.

7. The composition of claim 1, wherein the amount of the bacteriophage strain is effective for reducing a concentration of endotoxins produced by the pathogen in the intestinal microbiome of the subject.

8. The composition of claim 7, wherein the endotoxins include lipopolysaccharides.

9. The composition of claim 1, wherein the bacteriophage strain is a mutant or a recombinant bacteriophage of an isolated bacteriophage strain capable of infecting and lysing the pathogen.

10. The composition of claim 1, wherein the bacteriophage strain is unable to infect other microorganisms or cells other than the pathogen or other pathogens from the species of the pathogen.

11. The composition of claim 1, wherein the bacteriophage strain has a genome devoid of a polynucleotide encoding for a toxin or virulence factor.

12. The composition of claim 1, wherein the composition or bacteriophage strain thereof is in dried form.

13. A process of preparing the composition of claim 1, comprising:
characterizing the bacteriophage strain by exposing the pathogen to the bacteriophage strain and detecting infection or lysis of the pathogen; and
combining the bacteriophage strain with the pharmaceutically acceptable excipient when infection or lysis of the pathogen is detected.

14. The process of claim 13, wherein characterizing includes exposing a second pathogen from the species of the pathogen to the bacteriophage strain and detecting infection or lysis of the second pathogen and the combining includes combining the bacteriophage strain with the pharmaceutically acceptable excipient when infection or lysis of the second pathogen by the bacteriophage strain is detected.

15. The process of claim 13, wherein characterizing includes exposing a second pathogen of a species different from the species of the pathogen with the bacteriophage strain and detecting infection or lysis of the second pathogen and the combining includes combining the bacteriophage strain with the pharmaceutically acceptable excipient when infection or lysis of the second pathogen by the bacteriophage strain is not detected.

16. The composition of claim 1, wherein the at least one bacteriophage strain is at least two different bacteriophage strains, where each bacteriophage strain has an infectivity specific to the species of the pathogen, wherein each bacteriophage strain has a genome with less than 100% nucleotide sequence identity to genome(s) of the other bacteriophage strain(s).

17. (canceled)

18. The composition of claim 1, wherein the at least one bacteriophage strain is a range from two to ten different bacteriophage strains, where each bacteriophage strain has an infectivity specific to the species of the pathogen.

19. A process of preparing the composition of claim 16 comprising:
characterizing bacteriophage strains by separately exposing the pathogen to each bacteriophage strain and detecting infection or lysis of the pathogen;
selecting the at least two different bacteriophage strains from the bacteriophage strains that infect or lyse the pathogen; and
combining the at least two different bacteriophage strains with the pharmaceutically acceptable excipient.

20. The composition of claim 1 further comprising an amount of a second bacteriophage strain effective for reducing a concentration of a second pathogen of a species different from the species of the pathogen and in the intestinal microbiome of the subject, wherein the second pathogen induces obesity, inflammation, or an obesity-associated metabolic disorder and the second bacteriophage strain has an infectivity specific to a species of the second pathogen.

21. The composition of claim 20, further comprising an amount of a third bacteriophage strain that has an infectivity specific to the species of the second pathogen, wherein the third bacteriophage strain has a genome with less than 100% nucleotide sequence identity to genome of the third bacteriophage strain.

22. (canceled)

23. (canceled)

24. A process of preparing the composition of claim 20 comprising:
characterizing the bacteriophage strain by exposing the pathogen to the bacteriophage strain and detecting infection or lysis of the pathogen;
characterizing the second bacteriophage strain by exposing the second pathogen to the second bacteriophage strain and detecting infection or lysis of the second pathogen; and
combining the bacteriophage strain and the second bacteriophage strain with the pharmaceutically acceptable excipient when infection or lysis of the pathogen and the second pathogen are detected.

25.-90. (canceled)
